(19) United States
(12) Patent Application Publication
(10) Pub. No.: US 2024/0309009 A1
(43) Pub. Date: Sep. 19, 2024

(54) Anxiolytic Compounds, Pharmaceutical Compositions, and Methods of Treating Anxiety and Other Disorders

(71) Applicant: Miralogx LLC, Tampa, FL (US)

(72) Inventor: Jonnie R. Williams, SR., Sarasota, FL (US)

(21) Appl. No.: 18/673,060

(22) Filed: May 23, 2024

Related U.S. Application Data
(63) Continuation-in-part of application No. PCT/US2023/061381, filed on Jan. 26, 2023.
(60) Provisional application No. 63/524,372, filed on Jun. 30, 2023.
(60) Provisional application No. 63/304,118, filed on Jan. 28, 2022.
(60) Provisional application No. 63/301,123, filed on Jan. 20, 2022.

(51) Int. Cl. C07D 487/04; C07D 471/22

(52) U.S. Cl. CPC C07D 487/04; C07D 471/22

(57) ABSTRACT

In some aspects, pharmaceutical compounds are disclosed which may be useful for treating anxiety and/or other disorders. In another aspect, a pharmaceutical composition includes a therapeutically effective amount of the compound and a pharmaceutically acceptable vehicle therefor. In yet another aspect, a method of treating anxiety, depression, obsessive-compulsive disorder, tobacco addiction, alcohol addiction, cocaine addiction, headache, or cancer-related or other end-of-life psychological distress involves administering the pharmaceutical composition to an individual in need thereof.

### CROSS-REFERENCE TO RELATED APPLICATIONS

This application is a continuation-in-part of International Application No. PCT/US2023/061381, filed Jan. 26, 2023, which claims priority under 35 U.S.C. § 119(e) to U.S. Application No. 63/304,118, filed Jan. 28, 2022, and U.S. Application No. 63/304, 123, filed Jan. 28, 2022. This application also claims priority under 35 U.S.C. § 119(e) to U.S. Application No. 63/524,372, filed Jun. 30, 2023. The disclosure of each of these applications is hereby incorporated by reference in its entirety.

### BACKGROUND

Psychedelic agents such as psilocybin have been investigated for use in the treatment of anxiety, depression, obsessive-compulsive disorder, smoking addiction, alcohol addiction, cocaine addiction, headache, and cancer-related or other end-of-life psychological distress.

Psilocybin is a naturally occurring psychedelic prodrug compound produced by more than 200 species of fungus. As a prodrug, psilocybin is quickly converted by the body to psilocin, which has mind-altering effects which may include euphoria, visual and mental hallucinations, changes in perception, a distorted sense of time, and perceived spiritual experiences. Possible adverse reactions from psilocybin include nausea and panic attacks.

N,N-Dimethyltryptamine (DMT) is a chemical substance that occurs in many plants and animals and which is both a derivative and a structural analog of tryptamine. It can be consumed as a psychedelic drug and has historically been prepared by various cultures for ritual purposes as an entheogen. DMT has a rapid onset, intense effects, and a relatively short duration of action. DMT can be inhaled, ingested, or injected and its effects depend on the dose, as well as its mode of administration. When inhaled or injected, the effects last a short period of time, about 5-15 minutes. Effects can last three hours or more when orally ingested along with a monoamine oxidase inhibitor (MAOI), such as the ayahuasca brew of many native Amazonian tribes. DMT can produce vivid “projections” of mystical experiences involving euphoria and dynamic hallucinations of geometric forms.

5-MeO-DMT (5-methoxy-N,N-dimethyltryptamine) is another psychedelic of the tryptamine class. It is found in a wide variety of plant species, and at least one toad species, the Sonoran Desert toad. Like its close relatives DMT and bufotenine (5-HO-DMT), it has been used as an entheogen in South America.

Tabernanthalog (TBG) is a synthetic analog of the psychedelic drug ibogaine. Unlike ibogaine, TBG is water-soluble and may be synthesized in a single step. Experiments with cell cultures and zebrafish showed that it is less toxic than ibogaine, which can cause heart attacks and has been responsible for several deaths.

TBG also was reported to increased formation of new dendrites (branches) in rat nerve cells, and of new spines on those dendrites, similar to the effect of ketamine, LSD, MDMA and DMT (the active component in the plant extract ayahuasca) on connections between nerve cells. A. Fell, “New Compound Related to Psychedelic Ibogaine Could Treat Addiction, Depression,” Human & Animal Health, Dec. 9, 2020, available at ucdavis.edu/news/new-compound-related-psychedelic-ibogaine-could-treat-addiction-depression/.

There remains a need for improved therapies for treating anxiety and other disorders. It would be particularly desirable to develop small-molecule compounds that can be prepared synthetically and which exhibit improved efficacy while avoiding one or more drawbacks associated with presently available therapies.

### SUMMARY

In one aspect, the present disclosure relates to a compound having a structure of Formula (I):

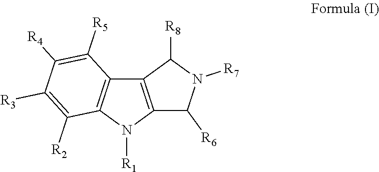

wherein R₁, R₂, R₃, R₄, R₅, R₆, R₇, and R₈ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula (I), wherein at least one of R₂, R₃, R₄, or R₅ is a phosphate.

In some embodiments, the present disclosure relates to a compound having a structure of Formula (I), wherein at least one of R₂, R₃, R₄, or R₅ is —O—R₉, preferably wherein R₃ is —O—R₉.

In some embodiments, the present disclosure relates to a compound having a structure of Formula (I), wherein R₉ is alkyl.

In some embodiments, the present disclosure relates to a compound having a structure of Formula (I), wherein R₉ is methyl.

In some embodiments, the present disclosure relates to a compound having a structure of Formula (I), wherein at least one of R₆, R₇, or R₈ is alkyl.

In some embodiments, the present disclosure relates to a compound having a structure of Formula (I), wherein at least one of R₆, R₇, or R₈ is methyl.

In some embodiments, the present disclosure relates to a compound having a structure of Formula (I), wherein R₇ is methyl.

In some embodiments, the present disclosure relates to a compound having a structure of Formula (I), wherein at least one of R₂, R₃, R₄, or R₅ is OH.

In some embodiments, the present disclosure relates to a compound having a structure of Formula (I), wherein at least one of R₅, R₆, R₇, and R₈ is alkyl.

In some embodiments, the present disclosure relates to a compound having a structure of Formula (I), wherein R₁ is alkyl.

In some embodiments, the present disclosure relates to a compound having a structure of Formula (I), wherein R₁ is H.

In some embodiments, the present disclosure relates to a compound having a structure of Formula (I), wherein R₁ and R₃ together form a carbocyclic or heterocyclic ring, optionally wherein the carbocyclic or heterocyclic ring is selected from a group consisting of an cycloalkane, aryl, cycloalkyl, heteroaryl, substituted cycloalkyl, cycloalkenyl, substituted cycloalkenyl, and heterocycle.

In some embodiments, the present disclosure relates to a compound having a structure of Formula (II):

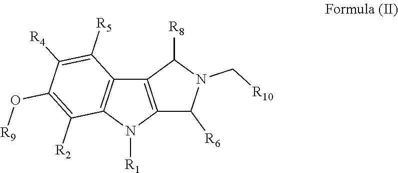

wherein R₁, R₂, R₄, R₅, R₈, R₉, and R₁₀ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula (II), wherein R₉ and R₁₀ together form a carbocyclic or heterocyclic ring, optionally wherein the carbocyclic or heterocyclic ring is selected from a group consisting of an cycloalkane, aryl, cycloalkyl, heteroaryl, substituted cycloalkyl, cycloalkenyl, substituted cycloalkenyl, and heterocycle.

In some embodiments, the present disclosure relates to a compound having a structure of Formula (I) or (II), wherein the compound is

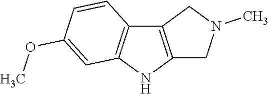

or a pharmaceutically acceptable salt or ester thereof.

In some embodiments, the present disclosure relates to a compound having a structure of Formula (III):

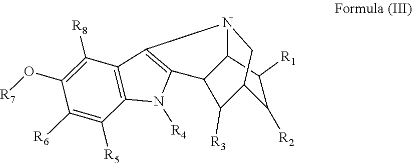

wherein R₁, R₂, R₃, R₄, R₅, R₆, R₇, and R₈ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula (III), wherein R₁ is alkyl.

In some embodiments, the present disclosure relates to a compound having a structure of Formula (III), wherein R₁ is C₁₋₄ alkyl.

In some embodiments, the present disclosure relates to a compound having a structure of Formula (III), wherein R₁ is ethyl.

In some embodiments, the present disclosure relates to a compound having a structure of Formula (III), wherein R₄ is H.

In some embodiments, the present disclosure relates to a compound having a structure of Formula (III), wherein R₇ is alkyl, preferably methyl.

In some embodiments, the present disclosure relates to a compound having a structure of Formula (III), wherein the compound is

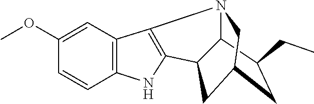

or a pharmaceutically acceptable salt or ester thereof.

In some embodiments, the present disclosure relates to a compound having a structure of Formula (IV):

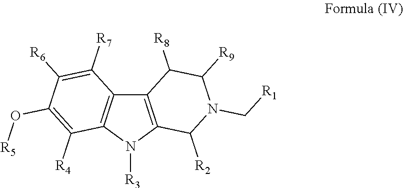

wherein R₁, R₂, R₃, R₄, R₅, R₆, R₇, R₈, and R₉ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula (IV), wherein R₁ is H.

In some embodiments, the present disclosure relates to a compound having a structure of Formula (IV), wherein R₃ is H.

In some embodiments, the present disclosure relates to a compound having a structure of Formula (IV), wherein R₅ is alkyl.

In some embodiments, the present disclosure relates to a compound having a structure of Formula (IV), wherein R₅ is methyl.

In some embodiments, the present disclosure relates to a compound having a structure of Formula (IV), wherein R₅ is H.

In some embodiments, the present disclosure relates to a compound having a structure of Formula (IV), wherein the compound is

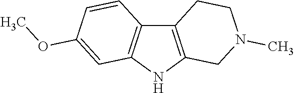

or a pharmaceutically acceptable salt or ester thereof.

In some embodiments, the present disclosure relates to a compound having a structure of Formula (V):

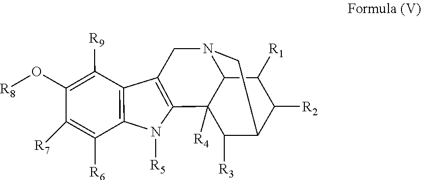

wherein R1, R2, R3, R4, R5, R6, R7, R8, and R9 are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula (V), wherein R₁ is alkyl.

In some embodiments, the present disclosure relates to a compound having a structure of Formula (V), wherein R₁ is ethyl.

In some embodiments, the present disclosure relates to a compound having a structure of Formula (V), wherein R₅ is H.

In some embodiments, the present disclosure relates to a compound having a structure of Formula (V), wherein R₈ is alkyl.

In some embodiments, the present disclosure relates to a compound having a structure of Formula (V), wherein R₈ is methyl.

In some embodiments, the present disclosure relates to a compound having a structure of Formula (V), wherein the compound is

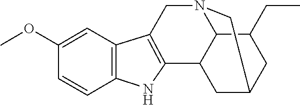 [SMILES: CCC1CC2CC3c4[nH]c5ccc(OC)cc5c4CN(C2)C13]

or a pharmaceutically acceptable salt or ester thereof.

In some embodiments, the present disclosure relates to a compound having a structure of Formula (VI):

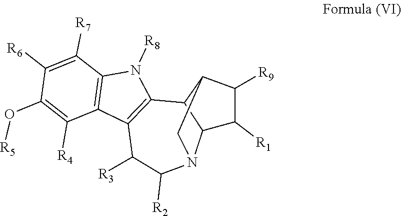

wherein R1, R2, R3, R4, R5, R6, R7, R8 and R9 are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula (VI), wherein R₁ is alkyl.

In some embodiments, the present disclosure relates to a compound having a structure of Formula (VI), wherein R₁ is ethyl.

In some embodiments, the present disclosure relates to a compound having a structure of Formula (VI), wherein R₈ is H.

In some embodiments, the present disclosure relates to a compound having a structure of Formula (VI), wherein R₅ is alkyl.

In some embodiments, the present disclosure relates to a compound having a structure of Formula (VI), wherein R₅ is methyl.

In some embodiments, the present disclosure relates to a compound having a structure of Formula (VI), wherein the compound is

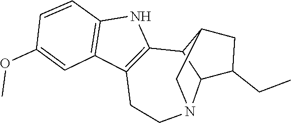 [SMILES: CCC1CC2CN3CCc4c([nH]c5ccc(OC)cc45)C2C13]

or a pharmaceutically acceptable salt or ester thereof.

In some embodiments, the present disclosure relates to a compound having a structure of Formula (VII):

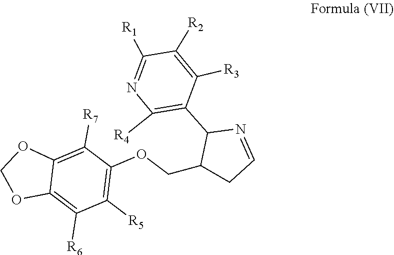

wherein R1, R2, R3, R4, R5, R6 and R7 are each independently selected from the group consisting of an electron pair, H, halogen, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula (VII), wherein at least one of R₁, R₂, R₃, or R₄ is an halogen.

In some embodiments, the present disclosure relates to a compound having a structure of Formula (VII), wherein R₁ is an halogen.

In some embodiments, the present disclosure relates to a compound having a structure of Formula (VII), wherein the halogen is selected from the group consisting of fluorine (F), chlorine (CI), bromine (Br), and iodine (I).

In some embodiments, the present disclosure relates to a compound having a structure of Formula (VII), wherein the halogen is F.

In some embodiments, the present disclosure relates to a compound having a structure of Formula (VII), wherein the compound is

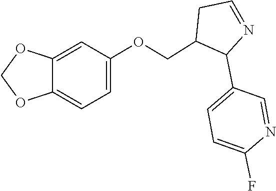 [SMILES: Fc1ccc(C2N=CCC2COc2ccc3c(c2)OCO3)cn1]

or a pharmaceutically acceptable salt or ester thereof.

In some embodiments, the present disclosure relates to a compound having a structure of Formula (VIII):

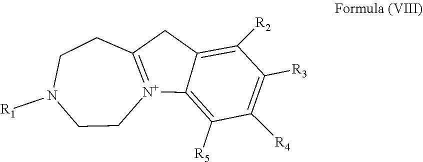

wherein R₁, R₂, R₃, R₄, and R₅ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula VIII, wherein at least one of R₂, R₃, R₄, or R₅ is —O—R₆.

In some embodiments, the present disclosure relates to a compound having a structure of Formula VIII, wherein R₃ is —O—R₆.

In some embodiments, the present disclosure relates to a compound having a structure of Formula VIII, wherein R₆ is alkyl.

In some embodiments, the present disclosure relates to a compound having a structure of Formula VIII, wherein R₆ is methyl.

In some embodiments, the present disclosure relates to a compound having a structure of Formula VIII, wherein the R₁ alkyl.

In some embodiments, the present disclosure relates to a compound having a structure of Formula VIII, wherein R₁ is methyl.

In some embodiments, the present disclosure relates to a compound having a structure of Formula VIII, wherein the compound is

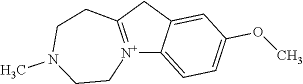

or a pharmaceutically acceptable salt or ester thereof.

In some embodiments, the present disclosure relates to a compound selected from the group consisting of 5-(3-((benzo[d][1,3]dioxol-5-yloxy)methyl)-3,4-dihydro-2H-pyrrol-2-yl)-2-fluoro pyridine, 9-methoxy-3-methyl-1,2,3,4,5,11-hexahydro-[1,4]diazepino[1,7-a]indol-6-ium, 6-methoxy-2-methyl-1,2,3,4-tetrahydropyrrolo[3,4-b]indole, (1S,3S,5S, 11R)-1-ethyl-7-methoxy-1,3,4,10,11,11a-hexahydro-2H-3,11-methanoindolizino[2,3-b]indole, 7-methoxy-2-methyl-2,3,4,9-tetrahydro-1H-pyrido[3,4-b]indole, 7-ethyl-2-methoxy-5,6,6a,7,8,9,10,12-octahydro-6,9-methanoindolo[2,3-b]quinolizine, 3-ethyl-8-methoxy-1,2,3,3a,5,6,11, 11b-octahydro-1,4-methanocyclopenta[2,3]azepino[4,5-b]indole, or a pharmaceutically acceptable salt, ester, or ether thereof.

In some embodiments, the present disclosure relates to a pharmaceutical composition comprising a therapeutically effective amount of the compound disclosed herein, and a pharmaceutically acceptable vehicle therefor.

In some embodiments, the present disclosure relates to a method of treating a disorder selected from the group consisting of anxiety, depression, obsessive-compulsive disorder, tobacco addiction, alcohol addiction, cocaine addiction, headache, and psychological distress, the method comprising administering to an individual in need thereof the compound or the pharmaceutical composition of disclosed herein.

In some embodiments, the present disclosure relates to a method of treating anxiety comprising administering to an individual in need thereof the compound or the pharmaceutical composition disclosed herein.

In some embodiments, the present disclosure relates to a method of treating a disorder selected from the group consisting of depression, obsessive-compulsive disorder, tobacco addiction, alcohol addiction, cocaine addiction, headache, and psychological distress, the method comprising administering to an individual in need thereof the compound or the pharmaceutical composition disclosed herein.

### DETAILED DESCRIPTION

The present disclosure introduces novel pharmaceutical compounds.

In some embodiments, the present disclosure relates to a compound having a structure of Formula (I):

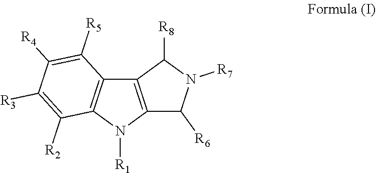

wherein R₁, R₂, R₃, R₄, R₅, R₆, R₇, and R₈ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula (I), wherein at least one of R₂, R₃, R₄, or R₅ is a phosphate.

In some embodiments, the present disclosure relates to a compound having a structure of Formula (I), wherein at least one of R₂, R₃, R₄, or R₅ is —O—R₉, preferably wherein R₃ is —O—R₉.

In some embodiments, the present disclosure relates to a compound having a structure of Formula (I), wherein R₉ is alkyl.

In some embodiments, the present disclosure relates to a compound having a structure of Formula (I), wherein R₉ is methyl.

In some embodiments, the present disclosure relates to a compound having a structure of Formula (I), wherein at least one of R₆, R₇, or R₈ is alkyl.

In some embodiments, the present disclosure relates to a compound having a structure of Formula (I), wherein at least one of R₆, R₇, or R₈ is methyl.

In some embodiments, the present disclosure relates to a compound having a structure of Formula (I), wherein R₇ is methyl.

In some embodiments, the present disclosure relates to a compound having a structure of Formula (I), wherein at least one of R₂, R₃, R₄, or R₅ is OH.

In some embodiments, the present disclosure relates to a compound having a structure of Formula (I), wherein at least one of R₅, R₆, R₇, and R₈ is alkyl.

In some embodiments, the present disclosure relates to a compound having a structure of Formula (I), wherein R₁ is alkyl.

In some embodiments, the present disclosure relates to a compound having a structure of Formula (I), wherein R₁ is H.

In some embodiments, the present disclosure relates to a compound having a structure of Formula (I), wherein R₁ and R₃ together form a carbocyclic or heterocyclic ring, optionally wherein the carbocyclic or heterocyclic ring is selected from a group consisting of an cycloalkane, aryl, cycloalkyl, heteroaryl, substituted cycloalkyl, cycloalkenyl, substituted cycloalkenyl, and heterocycle.

In some embodiments, the present disclosure relates to a compound having a structure of Formula (II):

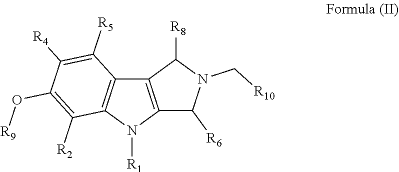

wherein R₁, R₂, R₄, R₅, R₈, R₉, and R₁₀ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula (II), wherein R₉ and R₁₀ together form a carbocyclic or heterocyclic ring, optionally wherein the carbocyclic or heterocyclic ring is selected from a group consisting of an cycloalkane, aryl, cycloalkyl, heteroaryl, substituted cycloalkyl, cycloalkenyl, substituted cycloalkenyl, and heterocycle.

In some embodiments, the present disclosure relates to a compound having a structure of Formula (I) or (II), wherein the compound is

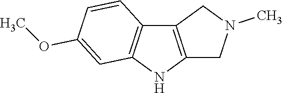

or a pharmaceutically acceptable salt or ester thereof.

In some embodiments, the present disclosure relates to a compound having a structure of Formula (III):

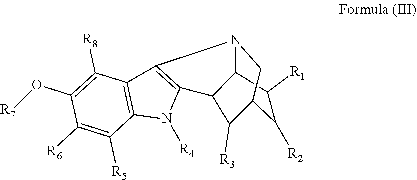

wherein R₁, R₂, R₃, R₄, R₅, R₆, R₇, and R₈ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein RA and RB are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula (III), wherein R₁ is alkyl.

In some embodiments, the present disclosure relates to a compound having a structure of Formula (III), wherein R₁ is C₁₋₄ alkyl.

In some embodiments, the present disclosure relates to a compound having a structure of Formula (III), wherein R₁ is ethyl.

In some embodiments, the present disclosure relates to a compound having a structure of Formula (III), wherein R₄ is H.

In some embodiments, the present disclosure relates to a compound having a structure of Formula (III), wherein R₇ is alkyl, preferably methyl.

In some embodiments, the present disclosure relates to a compound having a structure of Formula (III), wherein the compound is

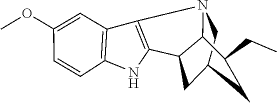

or a pharmaceutically acceptable salt or ester thereof.

In some embodiments, the present disclosure relates to a compound having a structure of Formula (IV):

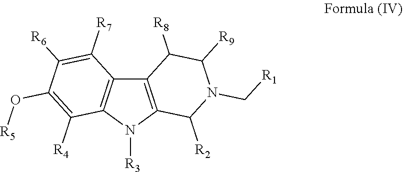

wherein R₁, R₂, R₃, R₄, R₅, R₆, R₇, R₈, and R₉ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula (IV), wherein R₁ is H.

In some embodiments, the present disclosure relates to a compound having a structure of Formula (IV), wherein R₃ is H.

In some embodiments, the present disclosure relates to a compound having a structure of Formula (IV), wherein R₅ is alkyl.

In some embodiments, the present disclosure relates to a compound having a structure of Formula (IV), wherein R₅ is methyl.

In some embodiments, the present disclosure relates to a compound having a structure of Formula (IV), wherein R₅ is H.

In some embodiments, the present disclosure relates to a compound having a structure of Formula (IV), wherein the compound is

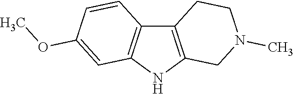

or a pharmaceutically acceptable salt or ester thereof.

In some embodiments, the present disclosure relates to a compound having a structure of Formula (V):

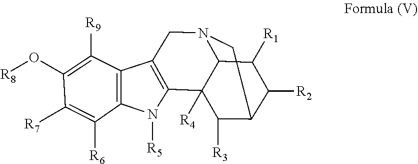

wherein R₁, R₂, R₃, R₄, R₅, R₆, R₇, R₈, and R₉ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula (V), wherein R₁ is alkyl.

In some embodiments, the present disclosure relates to a compound having a structure of Formula (V), wherein R₁ is ethyl.

In some embodiments, the present disclosure relates to a compound having a structure of Formula (V), wherein R₅ is H.

In some embodiments, the present disclosure relates to a compound having a structure of Formula (V), wherein R₈ is alkyl.

In some embodiments, the present disclosure relates to a compound having a structure of Formula (V), wherein R₈ is methyl.

In some embodiments, the present disclosure relates to a compound having a structure of Formula (V), wherein the compound is

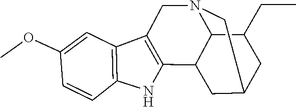 [SMILES: CCC1CC2CC3c4[nH]c5ccc(OC)cc5c4CN(C2)C13]

or a pharmaceutically acceptable salt or ester thereof.

In some embodiments, the present disclosure relates to a compound having a structure of Formula (VI):

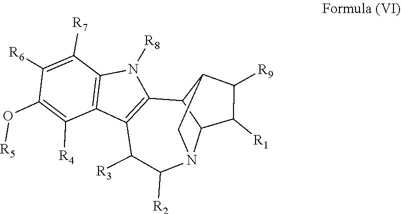

wherein R1, R2, R3, R4, R5, R6, R7, R8 and R9 are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula (VI), wherein R₁ is alkyl.

In some embodiments, the present disclosure relates to a compound having a structure of Formula (VI), wherein R₁ is ethyl.

In some embodiments, the present disclosure relates to a compound having a structure of Formula (VI), wherein R₈ is H.

In some embodiments, the present disclosure relates to a compound having a structure of Formula (VI), wherein R₅ is alkyl.

In some embodiments, the present disclosure relates to a compound having a structure of Formula (VI), wherein R₅ is methyl.

In some embodiments, the present disclosure relates to a compound having a structure of Formula (VI), wherein the compound is

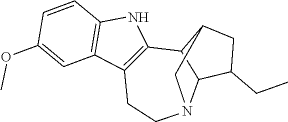 [SMILES: CCC1CC2CN3CCc4c([nH]c5ccc(OC)cc45)C2C13]

or a pharmaceutically acceptable salt or ester thereof.

In some embodiments, the present disclosure relates to a compound having a structure of Formula (VII):

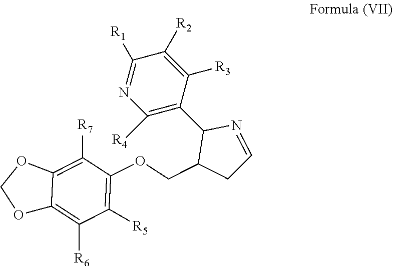

wherein R1, R2, R3, R4, R5, R6 and R7 are each independently selected from the group consisting of an electron pair, H, halogen, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and—SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula (VII), wherein at least one of R₁, R₂, R₃, or R₄ is an halogen.

In some embodiments, the present disclosure relates to a compound having a structure of Formula (VII), wherein R₁ is an halogen.

In some embodiments, the present disclosure relates to a compound having a structure of Formula (VII), wherein the halogen is selected from the group consisting of fluorine (F), chlorine (Cl), bromine (Br), and iodine (I).

In some embodiments, the present disclosure relates to a compound having a structure of Formula (VII), wherein the halogen is F.

In some embodiments, the present disclosure relates to a compound having a structure of Formula (VII), wherein the compound is

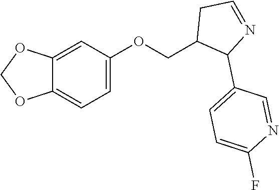 [SMILES: Fc1ccc(C2N=CCC2COc2ccc3c(c2)OCO3)cn1]

or a pharmaceutically acceptable salt or ester thereof.

In some embodiments, the present disclosure relates to a compound having a structure of Formula (VIII):

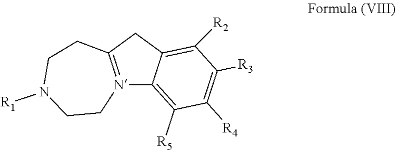

wherein R₁, R₂, R₃, R₄, and R₅ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, the present disclosure relates to a compound having a structure of Formula VIII, wherein at least one of R₂, R₃, R₄, or R₅ is —O—R₆.

In some embodiments, the present disclosure relates to a compound having a structure of Formula VIII, wherein R₃ is —O—R₆.

In some embodiments, the present disclosure relates to a compound having a structure of Formula VIII, wherein R₆ is alkyl.

In some embodiments, the present disclosure relates to a compound having a structure of Formula VIII, wherein R₆ is methyl.

In some embodiments, the present disclosure relates to a compound having a structure of Formula VIII, wherein the R₁ is alkyl.

In some embodiments, the present disclosure relates to a compound having a structure of Formula VIII, wherein R₁ is methyl.

In some embodiments, the present disclosure relates to a compound having a structure of Formula VIII, wherein the compound is

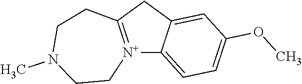

or a pharmaceutically acceptable salt or ester thereof.

In some embodiments, the present disclosure relates to a compound selected from the group consisting of 5-(3-((benzo[d][1,3]dioxol-5-yloxy)methyl)-3,4-dihydro-2H-pyrrol-2-yl)-2-fluoro pyridine, 9-methoxy-3-methyl-1,2,3,4,5,11-hexahydro-[1,4]diazepino[1,7-a]indol-6-ium, 6-methoxy-2-methyl-1,2,3,4-tetrahydropyrrolo[3,4-b]indole, (1S,3S,5S, 11R)-1-ethyl-7-methoxy-1,3,4,10,11,11a-hexahydro-2H-3,11-methanoindolizino[2,3-b]indole, 7-methoxy-2-methyl-2,3,4,9-tetrahydro-1H-pyrido[3,4-b]indole, 7-ethyl-2-methoxy-5,6,6a,7,8,9,10,12-octahydro-6,9-methanoindolo[2,3-b]quinolizine, 3-ethyl-8-methoxy-1,2,3,3a,5,6,11, 11b-octahydro-1,4-methanocyclopenta[2,3]azepino[4,5-b]indole, or a pharmaceutically acceptable salt, ester, or ether thereof.

In one example, a compound has the structure:

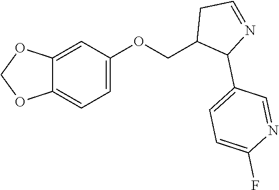 [SMILES: Fc1ccc(C2N=CCC2COc2ccc3c(c2)OCO3)cn1]

5-(3-((benzo[d][1,3]dioxol-5-yloxy)methyl)-3,4-dihydro-2H-pyrrol-2-yl)-2-fluoro pyridine, or a pharmaceutically acceptable salt, ester, or ether thereof.

In one example, the compound is 5-(3-((benzo[d][1,3]dioxol-5-yloxy)methyl)-3,4-dihydro-2H-pyrrol-2-yl)-2-fluoropyridine.

According to one aspect, a pharmaceutical compound has the structure:

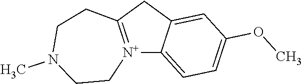

9-methoxy-3-methyl-1,2,3,4,5,11-hexahydro-[1,4]diazepino[1,7-a]indol-6-ium, or a pharmaceutically acceptable salt, ester, or ether thereof. In one example, the compound is 9-methoxy-3-methyl-1,2,3,4,5,11-hexahydro-[1,4]diazepino[1,7-a]indol-6-ium.

According to one aspect, a pharmaceutical compound has the structure:

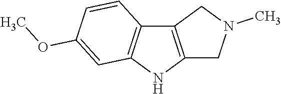

6-methoxy-2-methyl-1,2,3,4-tetrahydropyrrolo[3,4-b]indole, or a pharmaceutically acceptable salt, ester, or ether thereof. In one example, the compound is 6-methoxy-2-methyl-1,2,3,4-tetrahydropyrrolo[3,4-b]indole.

According to one aspect, a pharmaceutical compound has the structure:

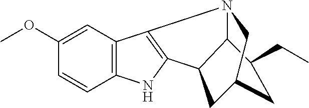

(1S,3S,5S,11R)-1-ethyl-7-methoxy-1,3,4,10,11,11a-hexahydro-2H-3,11-methanoindolizino[2,3-b]indole, or a pharmaceutically acceptable salt, ester, or ether thereof. In one example, the compound is (1S,3S,5S,11R)-1-ethyl-7-methoxy-1,3,4,10,11,11a-hexahydro-2H-3,11-methanoindolizino[2,3-b]indole.

According to one aspect, a pharmaceutical compound has the structure:

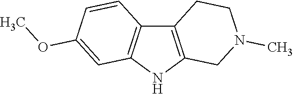

7-methoxy-2-methyl-2,3,4,9-tetrahydro-1H-pyrido[3,4-b]indole or a pharmaceutically acceptable salt, ester, or ether thereof. In one example, the compound is 7-methoxy-2-methyl-2,3,4,9-tetrahydro-1H-pyrido[3,4-b]indole.

According to one aspect, a pharmaceutical compound has the structure:

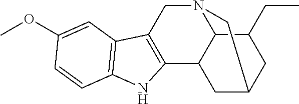 [SMILES: CCC1CC2CC3c4[nH]c5ccc(OC)cc5c4CN(C2)C13]

7-ethyl-2-methoxy-5,6,6a,7,8,9,10,12-octahydro-6,9-methanoindolo[2,3-b]quinolizine or a pharmaceutically acceptable salt, ester, or ether thereof. In one example, the compound is 7-ethyl-2-methoxy-5,6,6a,7,8,9,10,12-octahydro-6,9-methanoindolo[2,3-b]quinolizine.

According to one aspect, a pharmaceutical compound has the structure:

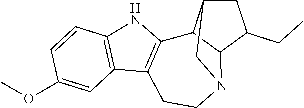 [SMILES: CCC1CC2CN3CCc4c([nH]c5ccc(OC)cc45)C2C13]

3-ethyl-8-methoxy-1,2,3,3a,5,6,11,11b-octahydro-1,4-methanocyclopenta[2,3]azepino [4,5-b]índole, or a pharmaceutically acceptable salt, ester, or ether thereof. In one example, the compound is 3-ethyl-8-methoxy-1,2,3,3a,5,6,11,11b-octahydro-1,4-methanocyclopenta[2,3]azepino[4,5-b]indole.

In some embodiments, a compound is selected from the group consisting of 5-(3-((benzo[d][1,3]dioxol-5-yloxy)methyl)-3,4-dihydro-2H-pyrrol-2-yl)-2-fluoro pyridine, 9-methoxy-3-methyl-1,2,3,4,5,11-hexahydro-[1,4]diazepino[1,7-a]indol-6-ium, 6-methoxy-2-methyl-1,2,3,4-tetrahydropyrrolo[3,4-b]indole, (1S,3S,5S,11R)-1-ethyl-7-methoxy-1,3,4,10,11,11a-hexahydro-2H-3,11-methanoindolizino[2,3-b]indole, 7- methoxy-2-methyl-2,3,4,9-tetrahydro-1H-pyrido[3,4-b]indole, 7-ethyl-2-methoxy-5,6,6a, 7,8,9,10, 12-octahydro-6,9-methanoindolo[2,3-b]quinolizine, 3-ethyl-8-methoxy-1,2,3,3a,5,6, 11,11b-octahydro-1,4-methanocyclopenta[2,3]azepino[4,5-b]indole, or a pharmaceutically acceptable salt, ester, or ether thereof.

In some embodiments, a compound has a structure of Formula (IX):

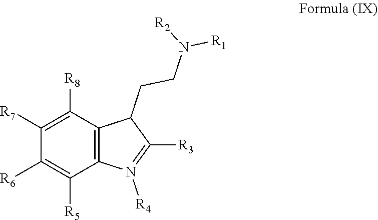

wherein R₁, R₂, R₃, R₄, R₅, R₆, R₇, and R₈ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof. In some embodiments, at least one of R₅, R₆, R₇, or R₈ is a phosphate.

In some embodiments, at least one of R₅, R₆, R₇, or R₈ is OH.

In some embodiments, at least one of R₅, R₆, R₇, and R₈ is —O-alkyl.

In some embodiments, at least one of R₁ and R₂ is alkyl.

In some embodiments, at least one of R₁ and R₂ is H.

In some embodiments, R₁ and R₃ together form a carbocyclic or heterocyclic ring, optionally wherein the carbocyclic or heterocyclic ring is selected from a group consisting of an cycloalkane, aryl, cycloalkyl, heteroaryl, substituted cycloalkyl, cycloalkenyl, substituted cycloalkenyl, and heterocycle.

In some embodiments, a compound has a structure of Formula (X):

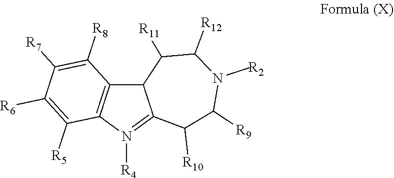

wherein R₂, R₄, R₅, R₆, R₇, and R₈ are as defined above, and R₉, R₁₀, R₁₁, and R₁₂ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

In some embodiments, R₉ and R₁₀ together form a carbocyclic or heterocyclic ring, optionally wherein the carbocyclic or heterocyclic ring is selected from a group consisting of an cycloalkane, aryl, cycloalkyl, heteroaryl, substituted cycloalkyl, cycloalkenyl, substituted cycloalkenyl, and heterocycle.

In some embodiments, a compound has a structure of Formula (XI):

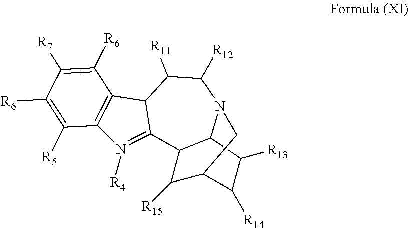

wherein R₄, R₅, R₆, R₇, and R₈ are defined as above, and wherein R₁₁, R₁₂, R₁₃, R₁₄ and R₁₅ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and—SO₂-alkyl; wherein R^{C} and R^{D} are each independently selected from hydrogen and C₁₋₄ alkyl; or a pharmaceutically acceptable salt or ester thereof.

In one example, a compound has the structure:

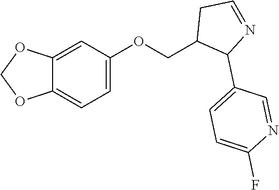 [SMILES: Fc1ccc(C2N=CCC2COc2ccc3c(c2)OCO3)cn1]

5-(3-((benzo[d][1,3]dioxol-5-yloxy)methyl)-3,4-dihydro-2H-pyrrol-2-yl)-2-fluoro pyridine, or a pharmaceutically acceptable salt, ester, or ether thereof. In one example, the compound is 5-(3-((benzo[d][1,3]dioxol-5-yloxy)methyl)-3,4-dihydro-2H-pyrrol-2-yl)-2-fluoropyridine.

According to one aspect, a pharmaceutical compound has the structure:

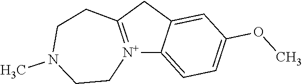

9-methoxy-3-methyl-1,2,3,4,5,11-hexahydro-[1,4]diazepino[1,7-a]indol-6-ium, or a pharmaceutically acceptable salt, ester, or ether thereof. In one example, the compound is 9-methoxy-3-methyl-1,2,3,4,5,11-hexahydro-[1,4]diazepino[1,7-a]indol-6-ium.

According to one aspect, a pharmaceutical compound has the structure:

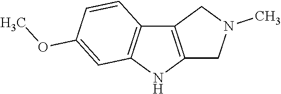

6-methoxy-2-methyl-1,2,3,4-tetrahydropyrrolo[3,4-b]indole, or a pharmaceutically acceptable salt, ester, or ether thereof. In one example, the compound is 6-methoxy-2-methyl-1,2,3,4-tetrahydropyrrolo[3,4-b]indole.

According to one aspect, a pharmaceutical compound has the structure:

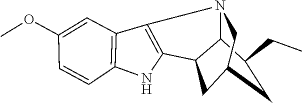

(1S,3S,5S,11R)-1-ethyl-7-methoxy-1,3,4,10,11,11a-hexahydro-2H-3,11-methanoindolizino[2,3-b]indole, or a pharmaceutically acceptable salt, ester, or ether thereof. In one example, the compound is (1S,3S,5S,11R)-1-ethyl-7-methoxy-1,3,4,10,11,11a-hexahydro-2H-3,11-methanoindolizino[2,3-b]indole.

According to one aspect, a pharmaceutical compound has the structure:

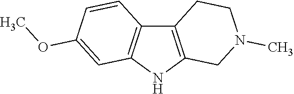

7-methoxy-2-methyl-2,3,4,9-tetrahydro-1H-pyrido[3,4-b]indole or a pharmaceutically acceptable salt, ester, or ether thereof. In one example, the compound is 7-methoxy-2-methyl-2,3,4,9-tetrahydro-1H-pyrido[3,4-b]indole.

According to one aspect, a pharmaceutical compound has the structure:

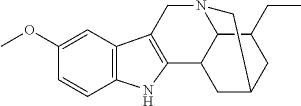 [SMILES: CCC1CC2CC3c4[nH]c5ccc(OC)cc5c4CN(C2)C13]

7-ethyl-2-methoxy-5,6,6a,7,8,9,10,12-octahydro-6,9-methanoindolo[2,3-b]quinolizine or a pharmaceutically acceptable salt, ester, or ether thereof. In one example, the compound is 7-ethyl-2-methoxy-5,6,6a,7,8,9,10,12-octahydro-6,9-methanoindolo[2,3-b]quinolizine.

According to one aspect, a pharmaceutical compound has the structure:

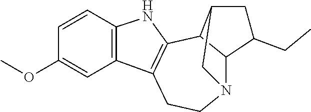 [SMILES: CCC1CC2CN3CCc4c([nH]c5ccc(OC)cc45)C2C13]

3-ethyl-8-methoxy-1,2,3,3a,5,6,11,11b-octahydro-1,4-methanocyclopenta[2,3]azepino [4,5-b]índole, or a pharmaceutically acceptable salt, ester, or ether thereof. In one example, the compound is 3-ethyl-8-methoxy-1,2,3,3a,5,6,11,11b-octahydro-1,4-methanocyclopenta[2,3]azepino[4,5-b]indole.

In some embodiments, a compound is selected from the group consisting of 5-(3-((benzo[d][1,3]dioxol-5-yloxy)methyl)-3,4-dihydro-2H-pyrrol-2-yl)-2-fluoro pyridine, 9-methoxy-3-methyl-1,2,3,4,5,11-hexahydro-[1,4]diazepino[1,7-a]indol-6-ium, 6-methoxy-2-methyl-1,2,3,4-tetrahydropyrrolo[3,4-b]indole, (1S,3S,5S,11R)-1-ethyl-7-methoxy-1,3,4,10,11,11a-hexahydro-2H-3,11-methanoindolizino[2,3-b]indole, 7-methoxy-2-methyl-2,3,4,9-tetrahydro-1H-pyrido[3,4-b]indole, 7-ethyl-2-methoxy-5,6,6a,7,8,9,10,12-octahydro-6,9-methanoindolo[2,3-b]quinolizine, 3-ethyl-8-methoxy-1,2,3,3a,5,6,11,11b-octahydro-1,4-methanocyclopenta[2,3]azepino[4,5-b]indole, or a pharmaceutically acceptable salt, ester, or ether thereof.

In one example, the compound has the structure:

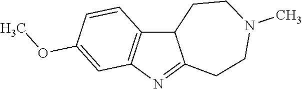

(8-methoxy-3-methyl-1,2,3,4,5,10b-hexahydroazepino[4,5-b]indole) or a pharmaceutically acceptable salt, ester, or ether thereof. In some embodiments, the compound is 8-methoxy-3-methyl-1,2,3,4,5,10b-hexahydroazepino[4,5-b]indole.

In another aspect, the compound has the structure:

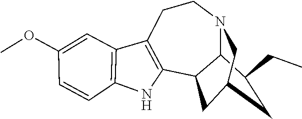 [SMILES: CC[C@H]1C[C@H]2C[C@H]3c4[nH]c5ccc(OC)cc5c4CC[N@@](C2)C13]

((6R,7S,9S,11S)-7-ethyl-2-methoxy-6,6a,7,9,10,12,13,13a-octahydro-8H-6,9-methanopyrido[1′,2′:1,2]azepino[4,5-b]indole), or a pharmaceutically acceptable salt, ester, or ether thereof. In some embodiments, the compound is (6R,7S,9S,11S)-7-ethyl-2-methoxy-6,6a,7,9,10,12,13,13a-octahydro-8H-6,9-methanopyrido[1′,2′:1,2]azepino[4,5-b]indole.

In one example, a pharmaceutical compound has a structure selected from the group consisting of:

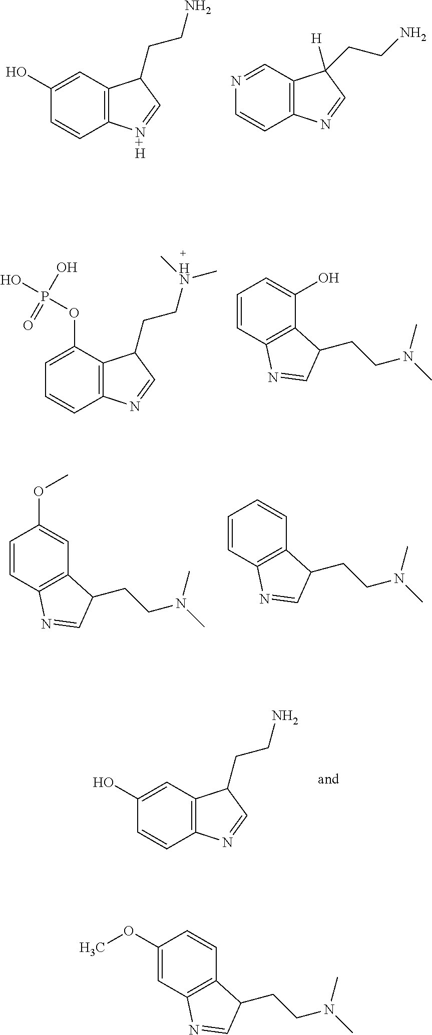

or a pharmaceutically acceptable salt, ester, or ether thereof.

In some embodiments. the compound has the structure

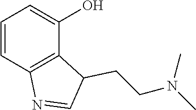

(3-(2-(dimethylamino)ethyl)-3H-indol-4-ol) or a pharmaceutically acceptable salt, ester, or ether thereof.

In some embodiments, the compound is selected from the group consisting of 3-(2-aminoethyl)-5-hydroxy-3H-indol-1-ium, 2-(3H-pyrrolo[3,2-c]pyridin-3-yl)ethan-1-amine, 3-(2-(dimethylamino)ethyl)-3H-indol-4-yl dihydrogen phosphate, 3-(2-(dimethylamino)ethyl)-3H-indol-4-ol, 2-(6-methoxy-3H-indol-3-yl)-N,N-dimethylethan-1-amine, 2-(3H-indol-3-yl)-N,N-dimethylethan-1-amine, 3-(2-aminoethyl)-3H-indol-5-ol, 2-(5-methoxy-3H-indol-3-yl)-N,N-dimethylethan-1-amine, or a pharmaceutically acceptable salt, ester, or ether thereof.

In some embodiments, the present disclosure relates to a pharmaceutical composition comprising a therapeutically effective amount of a compound disclosed herein, and a pharmaceutically acceptable vehicle therefor.

In some embodiments, the present disclosure relates to a method of treating a disorder selected from the group consisting of anxiety, depression, obsessive-compulsive disorder, tobacco addiction, alcohol addiction, cocaine addiction, headache, and psychological distress, the method comprising administering to an individual in need thereof the compound or the pharmaceutical composition of disclosed herein.

In some embodiments, the present disclosure relates to a method of treating anxiety comprising administering to an individual in need thereof the compound or the pharmaceutical composition disclosed herein.

In some embodiments, the present disclosure relates to a method of treating a disorder selected from the group consisting of depression, obsessive-compulsive disorder, tobacco addiction, alcohol addiction, cocaine addiction, headache, and psychological distress, the method comprising administering to an individual in need thereof the compound or the pharmaceutical composition disclosed herein.

A pharmaceutical composition may include a pharmaceutically acceptable carrier that facilitates processing of an active ingredient into pharmaceutically acceptable compositions. As used herein, the term “pharmacologically acceptable carrier” is synonymous with “pharmacological carrier” and means any carrier that has substantially no long term or permanent detrimental effect when administered and encompasses terms such as “pharmacologically acceptable vehicle,” “stabilizer,” “diluent,” “additive,” “auxiliary” or “excipient.” Such a carrier generally is mixed with an active compound or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7th ed. 1999); REMINGTON: THE SCIENCE AND PRACTICE OF PHARMACY (Alfonso R. Gennaro ed., Lippincott, Williams & Wilkins, 20th ed. 2000); Goodman & Gilman's The Pharmacological Basis of Therapeutics (Joel G. Hardman et al., eds., McGraw-Hill Professional, 10th ed. 2001); and Handbook of Pharmaceutical Excipients (Raymond C. Rowe et al., APhA Publications, 4th edition 2003). These protocols are routine procedures and any modifications are well within the scope of one skilled in the art and from the teaching hercin.

Compounds intended for administration to humans or other mammals generally should have very high purity. Purity refers to the ratio of a compound's mass to the total sample mass following any purification steps. Usually, the level of purity is at least about 95%, more usually at least about 96%, about 97%, about 98%, or higher. For example, the level of purity may be about 98.5%, 99.0%, 99.1%, 99.2%, 99.3%, 99.4%, 99.5%, 99.6%, 99.7%, 99.8%, 99.9%, or higher.

The compounds described herein that exist in more than one optical isomer form (enantiomer) may be provided either as racemic mixture or by isolating one of the enantiomers, the latter case in which purity as described above may refer to enantiomeric purity.

The compounds may be prepared synthetically using techniques described, for example, in L. P. Cameron et al., “A non-hallucinogenic psychedelic analogue with therapeutic potential,” Nature 589, 474-479 (2021), doi.org/10.1038/s41586-020-3008-z, with appropriate modifications of reagents to obtain the structures described herein as will be apparent to persons skilled in the art.

The compounds described herein may be prepared synthetically using techniques described, for example, in O. Shirota et al., “Concise Large-Scale Synthesis of Psilocin and Psilocybin, Principal Hallucinogenic Constituents of ‘Magic Mushroom,’” J. Nat. Prod. 2003, 66, 6, 885-887 (2003), doi.org/10.1021/np030059u, with appropriate modifications of reagents to obtain the structures described herein as will be apparent to persons skilled in the art. DMT derivatives may be synthesized through the reaction of indole with oxalyl chloride followed by reaction with dimethylamine and reduction of the carbonyl functionalities with lithium aluminum hydride. Another route is through the N,N-dimethylation of tryptamine using formaldehyde followed by reduction with sodium cyanoborohydride or sodium triacetoxyborohydride.

In some aspects, the compounds may be converted into a pharmaceutically acceptable salts using techniques well known to persons skilled in the art. For example, salts such as sodium and potassium salts may be prepared by treating the compound with a suitable sodium or potassium base, such as sodium hydroxide or potassium hydroxide, respectively. Esters and ethers of the compounds may be prepared as described, e.g., in Advanced Organic Chemistry, 1992, 4th Edition, J. March, John Wiley & Sons, or J. Med. Chemistry, 1992, 35, 145-151.

The compounds as described herein may be useful for treating anxiety, depression, obsessive-compulsive disorder, tobacco addiction (including smoking addiction and smokeless tobacco addiction), alcohol addiction, cocaine addiction, headache, and cancer-related or other end-of-life psychological distress.

Compositions as described herein may be administered orally, nasally, topically, subcutaneously, intramuscularly, intravenously, or by other suitable modes of administration.

A pharmaceutical composition may optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition.

Examples of auxiliaries and/or excipients that may be mentioned are cremophor, poloxamer, benzalkonium chloride, sodium lauryl sulfate, dextrose, glycerin, magnesium stearate, polyethylene glycol, starch, dextrin, lactose, cellulose, carboxymethylcellulose sodium, talc, agar-agar, mineral oil, animal oil, vegetable oil, organic and mineral waxes, paraffin, gels, propylene glycol, benzyl alcohol, dimethylacetamide, ethanol, polyglycols, tween 80, solutol HS 15, and water. It is also possible to administer the active substances as such, without vehicles or diluents, in a suitable form, for example, in capsules.

A pharmaceutical composition may comprise a therapeutic compound in an amount sufficient to allow customary administration to an individual. A unit dose form may have, e.g., at least 0.001 mg, at least 0.005 mg, at least 0.01 mg, at least 0.1 mg, at least 0.5 mg, at least 0.75 mg, at least 1 mg, at least 1.5 mg, at least 2 mg, at least 2.5 mg, at least 5 mg, at least 7.5 mg, at least 10 mg, at least 15 mg, at least 20 mg, at least 25 mg, at least 30 mg, at least 40 mg, or at least 50 mg of a therapeutic compound. In yet other aspects of this embodiment, a pharmaceutical composition disclosed herein may include, e.g., about 0.001 mg to about 500 mg, about 0.01 mg to about 250 mg, or about 0.05 mg to about 100 mg of a therapeutic compound.

Pharmaceutical compositions as described herein may include a pharmaceutically acceptable solvent. A solvent is a liquid, solid, or gas that dissolves another solid, liquid, or gaseous (the solute), resulting in a solution. Solvents useful in the pharmaceutical compositions include, without limitation, a pharmaceutically acceptable polar aprotic solvent, a pharmaceutically acceptable polar protic solvent and a pharmaceutically acceptable non-polar solvent. A pharmaceutically acceptable polar aprotic solvent includes, without limitation, dichloromethane (DCM), tetrahydrofuran (THF), ethyl acetate, acetone, dimethylformamide (DMF), acetonitrile (MeCN), dimethyl sulfoxide (DMSO). A pharmaceutically acceptable polar protic solvent includes, without limitation, acetic acid, formic acid, ethanol, n-butanol, 1-butanol, 2-butanol, isobutanol, sec-butanol, tert-butanol, n-propanol, isopropanol, 1,2 propan-diol, methanol, glycerol, and water. A pharmaceutically acceptable non-polar solvent includes, without limitation, pentane, cyclopentane, hexane, cyclohexane, benzene, toluene, 1,4-dioxane, chloroform, n-methyl-pyrrolidone (NMP), and diethyl ether.

The method of administration as well as the dosage range which are suitable in a specific case depend on the species to be treated and on the state of the respective condition or disease, and may be optimized using techniques known in the art. In some aspects, microdosing techniques may be used as described, e.g., in L. P. Cameron et al., “Chronic, Intermittent Microdoses of the Psychedelic N,N-Dimethyltryptamine (DMT) Produce Positive Effects on Mood and Anxiety in Rodents,” ACS Chem. Neurosci. 2019, 10, 3261-70.

By way of non-limiting example, the daily dose of an active compound may be about 0.0001 mg to about 10 mg per kg, or from about 0.0005 mg to about 5 mg per kg. Dosing can be single dosage or cumulative (serial dosing), and can be readily determined by one skilled in the art. For instance, treatment of anxiety may comprise a one-time administration of an effective dose of a pharmaceutical composition as disclosed herein. Alternatively, treatment may comprise multiple administrations of an effective dose of a pharmaceutical composition carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a pharmaceutical composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a pharmaceutical composition disclosed herein that is administered can be adjusted accordingly.

Pharmaceutical compositions may contain any conventional non-toxic pharmaceutically acceptable carriers, adjuvants or vehicles. In some cases, the pH of the formulation may be adjusted with acceptable pharmaceutical or food grade acids, bases or buffers to enhance the stability of the formulated composition or its delivery form.

Liquid dosage forms for oral administration include acceptable pharmaceutical or food grade emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylsulfoxide (DMSO) dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, lozenges, pills, powders, and granules. In such solid dosage forms, the active compound is mixed with at least one inert, acceptable pharmaceutical or food grade excipient or carrier such as sodium citrate or dicalcium phosphate and/or a) fillers or extenders such as starches, lactose, sucrose, glucose, mannitol, and silicic acid, b) binders such as carboxymethylcellulose, alginates, gelatin, polyvinylpyrrolidone, sucrose and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as cetyl alcohol and glycerol monostearate, h) absorbents such as kaolin and bentonite clay, i) lubricants such as talc, calcium stearate, magnesium stearate, solid polyethylene glycols, sodium lauryl sulfate, and mixtures thereof, and j) sweetening, flavoring, perfuming agents, and mixtures thereof. In the case of capsules, lozenges, tablets and pills, the dosage form may also comprise buffering agents.

The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract or, optionally, in a delayed or extended manner. Examples of embedding compositions which can be used include polymeric substances and waxes. Tablet formulations for extended release are also described in U.S. Pat. No. 5,942,244.

Compositions may contain a compound as disclosed herein, alone or with other therapeutic compound(s). A therapeutic compound is a compound that provides pharmacological activity or other direct effect in the diagnosis, cure, mitigation, treatment, or prevention of disease, or to affect the structure or any function of the body of man or animals. A therapeutic compound disclosed herein may be used in the form of a pharmaceutically acceptable salt, solvate, or solvate of a salt, e.g., a hydrochloride. Additionally, therapeutic compound disclosed herein may be provided as racemates, or as individual enantiomers, including the R-or S-enantiomer. Thus, the therapeutic compound disclosed herein may comprise a R-enantiomer only, a S-enantiomer only, or a combination of both a R-enantiomer and a S-enantiomer of a therapeutic compound. In some aspects, the therapeutic compound may have anti-inflammatory activity, such as a non-steroidal anti-inflammatory drug (NSAID). NSAIDs are a large group of therapeutic compounds with analgesic, anti-inflammatory, and anti-pyretic properties. NSAIDs reduce inflammation by blocking cyclooxygenase. NSAIDs include, without limitation, aceclofenac, acemetacin, actarit, alcofenac, alminoprofen, amfenac, aloxipirin, aminophenazone, antraphenine, aspirin, azapropazone, benorilate, benoxaprofen, benzydamine, butibufen, celecoxib, chlorthenoxacin, choline salicylate, clometacin, dexketoprofen, diclofenac, diflunisal, emorfazone, epirizole; ctodolac, etoricoxib, feclobuzone, felbinac, fenbufen, fenclofenac, flurbiprofen, glafenine, hydroxylethyl salicylate, ibuprofen, indometacin, indoprofen, ketoprofen, ketorolac, lactyl phenetidin, loxoprofen, lumiracoxib, mefenamic acid, meloxicam, metamizole, metiazinic acid, mofebutazone, mofezolac, nabumetone, naproxen, nifenazone, niflumic acid, oxametacin, phenacetin, pipebuzone, pranoprofen, propyphenazone, proquazone, protizinic acid, rofecoxib, salicylamide, salsalate, sulindac, suprofen, tiaramide, tinoridine, tolfenamic acid, valdecoxib, and zomepirac.

NSAIDs may be classified based on their chemical structure or mechanism of action. Non-limiting examples of NSAIDs include a salicylate derivative NSAID, a p-amino phenol derivative NSAID, a propionic acid derivative NSAID, an acetic acid derivative NSAID, an enolic acid derivative NSAID, a fenamic acid derivative NSAID, a non-selective cyclooxygenase (COX) inhibitor, a selective cyclooxygenase-1 (COX-1) inhibitor, and a selective cyclooxygenase-2 (COX-2) inhibitor. An NSAID may be a profen. Examples of a suitable salicylate derivative NSAID include, without limitation, acetylsalicylic acid (aspirin), diflunisal, and salsalate. Examples of a suitable p-amino phenol derivative NSAID include, without limitation, paracetamol and phenacetin. Examples of a suitable propionic acid derivative NSAID include, without limitation, alminoprofen, benoxaprofen, dexketoprofen, fenoprofen, flurbiprofen, ibuprofen, indoprofen, ketoprofen, loxoprofen, naproxen, oxaprozin, pranoprofen, and suprofen. Examples of a suitable acetic acid derivative NSAID include, without limitation, aceclofenac, acemetacin, actarit, alcofenac, amfenac, clometacin, diclofenac, etodolac, felbinac, fenclofenac, indometacin, ketorolac, metiazinic acid, mofezolac, nabumetone, naproxen, oxametacin, sulindac, and zomepirac. Examples of a suitable enolic acid (oxicam) derivative NSAID include, without limitation, droxicam, isoxicam, lornoxicam, meloxicam, piroxicam, and tenoxicam. Examples of a suitable fenamic acid derivative NSAID include, without limitation, flufenamic acid, mefenamic acid, meclofenamic acid, and tolfenamic acid. Examples of a suitable selective COX-2 inhibitors include, without limitation, celecoxib, etoricoxib, firocoxib, lumiracoxib, meloxicam, parecoxib, rofecoxib, and valdecoxib.

In one aspect, the present disclosure relates to a method of treating a disorder selected from the group consisting of anxiety, depression, obsessive-compulsive disorder, tobacco addiction, alcohol addiction, cocaine addiction, headache, and psychological distress, the method comprising administering to an individual in need thereof a pharmaceutical composition disclosed herein.

In one aspect, the present disclosure relates to a method of treating anxiety comprising administering to an individual in need thereof a pharmaceutical composition disclosed herein.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

While the invention has been described with respect to specific examples, those skilled in the art will appreciate that there are numerous variations and permutations of the above described systems and techniques that fall within the spirit and scope of the invention as set forth in the appended claims.

What is claimed is:

1. A compound having a structure of Formula (I): 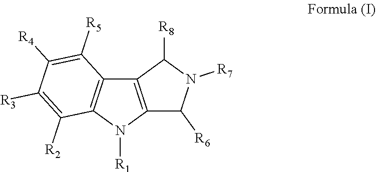
wherein R₁, R₂, R₃, R₄, R₅, R₆, R₇, and R₈ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

2. The compound of claim 1, wherein at least one of R₂, R₃, R₄, or R₅ is a phosphate.

3. The compound of claim 2, wherein at least one of R₂, R₃, R₄, or R₅ is —O—R₉, wherein R₉ is alkyl.

4. The compound of claim 3, wherein R₉ is methyl.

5. The compound of claim 1, wherein at least one of R₆, R₇, or R₈ is alkyl.

6. The compound of claim 1, wherein at least one of R₆, R₇, or R₈ is methyl.

7. The compound of claim 1, wherein R₇ is methyl.

8. The compound of claim 1, wherein at least one of R₂, R₃, R₄, or R₅ is OH.

9. The compound of claim 1, wherein at least one of R₅, R₆, R₇, and R₈ is alkyl.

10. The compound of claim 1, wherein R₁ is alkyl.

11. The compound of claim 1, wherein R₁ is H.

12. The compound of claim 1, wherein R₁ and R₃ together form a carbocyclic or heterocyclic ring, optionally wherein the carbocyclic or heterocyclic ring is selected from a group consisting of an cycloalkane, aryl, cycloalkyl, heteroaryl, substituted cycloalkyl, cycloalkenyl, substituted cycloalkenyl, and heterocycle.

13. The compound of claim 1, wherein the compound has a structure of Formula (II): 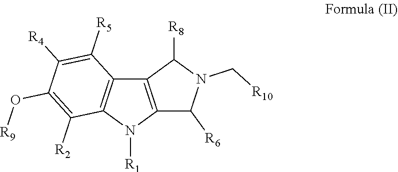
wherein R₉ and R₁₀ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

14. The compound of claim 13, wherein R₉ and R₁₀ together form a carbocyclic or heterocyclic ring, optionally wherein the carbocyclic or heterocyclic ring is selected from a group consisting of an cycloalkane, aryl, cycloalkyl, heteroaryl, substituted cycloalkyl, cycloalkenyl, substituted cycloalkenyl, and heterocycle.

15. The compound of claim 13, wherein the compound is 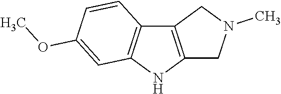
or a pharmaceutically acceptable salt or ester thereof.

16. A compound having a structure of Formula (III): 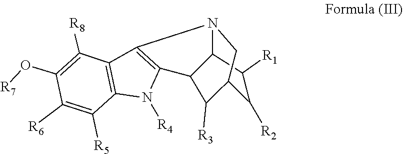
wherein R₁, R₂, R₃, R₄, R₅, R₆, R₇, and R₈ are each independently selected from the group consisting of an electron pair, H, OH, protected hydroxyl, alkyl, alkenyl, alkynyl, acyl, aryl, heteroaryl, cycloalkyl, phenyl, carbonate ester, a carboxylate, a carboxyl, an ester, a hydroperoxy, a peroxy, an ether, a hemiacetal, a hemiketal, an acetal, a ketal, an orthoester, a methylenedioxy, an orthocarbonate ester, carboxamide, an amine, an imine, an amide, an azide, an azo, a cyanate, a nitrate, a nitrile, an isonitrile, a nitrosooxy, a nitro, a pyridyl, a thiol, a sulfide, sulfinyl, a sulfonyl, a thiocyanate, a carbonothioyl, a phosphate, and heterocycle; optionally wherein the alkyl, alkenyl, alkynyl or acyl is substituted with one or more substituents independently selected from the group consisting of halogen, —OH, alkyl, —O-alkyl, NR^{A}R^{B}, —S-alkyl, —SO-alkyl, —SO₂-alkyl, alkenyl, alkynyl, aryl, heteroaryl, cycloalkyl, and heterocycle; wherein R^{A} and R^{B} are each independently selected from hydrogen and C₁₋₄ alkyl; wherein the aryl or heteroaryl, whether alone or as part of a substituent group, is optionally substituted with one or more substituents independently selected from the group consisting of halogen, —OH, alkyl, —O-alkyl, —COOH, —C(O)—C₁₋₄ alkyl, —C(O)O—C₁₋₄ alkyl, NR^{C}R^{D}, —S-alkyl, —SO-alkyl and —SO₂-alkyl; wherein R^{C} and R^{D} are each independently selected from hydrogen and C₁₋₄ alkyl; or a pharmaceutically acceptable salt or ester thereof.

17. The compound of claim 16, wherein the compound is 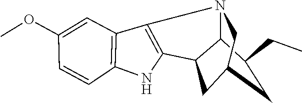
or a pharmaceutically acceptable salt or ester thereof.

18. A compound selected from the group consisting of 5-(3-((benzo[d][1,3]dioxol-5-yloxy)methyl)-3,4-dihydro-2H-pyrrol-2-yl)-2-fluoro pyridine, 9-methoxy-3-methyl-1,2,3,4,5,11-hexahydro-[1,4]diazepino[1,7-a]indol-6-ium, 6-methoxy-2-methyl-1,2,3,4-tetrahydropyrrolo[3,4-b]indole, (1S,3S,5S,11R)-1-ethyl-7-methoxy-1,3,4,10,11,11a-hexahydro-2H-3,11-methanoindolizino[2,3-b]indole, 7-methoxy-2-methyl-2,3,4,9-tetrahydro-1H-pyrido[3,4-b]indole, 7-ethyl-2-methoxy-5,6,6a,7,8,9,10,12-octahydro-6,9-methanoindolo[2,3-b]quinolizine, 3-ethyl-8-methoxy-1,2,3,3a,5,6,11,11b-octahydro-1,4-methanocyclopenta[2,3]azepino[4,5-b]indole, or a pharmaceutically acceptable salt, ester, or ether thereof.

19. A pharmaceutical composition comprising a therapeutically effective amount of the compound of claim 18, and a pharmaceutically acceptable vehicle therefor.

20. A method of treating a disorder selected from the group consisting of anxiety, depression, obsessive-compulsive disorder, tobacco addiction, alcohol addiction, cocaine addiction, headache, and psychological distress, the method comprising administering to an individual in need thereof the pharmaceutical composition of claim 19.
